SEQUENCE LISTING (5 sequences)

SEQ ID NO: 1 - protein, 1318 aa, Clostridium ljungdahlii
MYFDKETDFEEAVIKALIECGWESEVIRHPSEKDLIKNWANILFNNNRQRDRLNECPLTDGEMQQIMEQIKLLQSPIKLNGFINGKSVSIKRDNPDDKEHFGKEVSLKIYDRQEIAYGESRYQIVQQPHFPTKSPILNNRRGDLMLLINGMPVIHIELKKSGIPVSEAYNQIEKYSHEGVFRGLFSLIQVFVAMQPEETVYFANPGEGVKFNKAFYFHWANFNNQPINKWDEVISKLLNIPMAHMIIGFYTIADTDEGVLKVMRSYQYYAAIGIADKVAKKNWKDNNQLGGHVWHTTGSGKTMTSFKSAQLIASSHDADKVIFLVDRKELGIQSLKEYRSFANESESVQATENTNILISKLKSDDLDNTLIVTSIQKLSNISEEAQELNEADLKIIQSKRIAIIIDECHRSTFGEMLTNIKNTFKHSIIFGFTGTPIQDVNIKKDSTTPTIFGDEIHRYTLADGIRDKNVLGFDPYMVKIYDDSDLRQAVALHKAKAATVEEVMGDPKKEKMFYKYMDSSQVKMYGEQVDGKWVAGIEDFIPNEQYQTAEYRNGVIADIKKKWTIYSRGRKFHAIFATSNIPEAVVYYRLMKAEMPNLSITAMFDPSIDNEGGGSLEKEDGIVEILEDYKAKFSQSFTIKSYDKFRKDVSLRLAHKKPYERITPDEQIDILIVVNQMLTGFDSKWVNTLYLDKVMEYENLIQAFSRTNRLFNVADKPFGIIKYYRHPNTMEKNIEAAVKAYSGDIPTGLFVDKLPNNLRQMNSLFDEIQILFKNASISNFERLPDESSVKAKFAKQFKQFSTHLEAATIQGFIWSQKTYSDENGTSIVMHLDEMTYLILLARYMELAKGGGGGRSGDVPYEIDTHITEYDTEKIDADYMNSRFEKFLKLIESEYDAESLDKTLKELHKSFSMLSQEEQKYAIIFIRDIQAGNIHVIPGKSFRNYISQMMKKAENDRIKRVVNRLGCYEKLLREMLQRKVTKETIEAHGKFDELKATVDNQKARTFFILVEQDNYQESRLAMYIEQYLRNFLLSGGEDPYSNVEYSEVIREKNESEYNASVGVVLTEEKMKGKSIVSSVKSATLNNWYSESKALACMLETDCFAYVENKLCIYDNKYIQRNQNGRMFLTNYAKEHEEECFLQFIIDNETGKLHYITLPAAMASKSFNYYDEINEDLLTQYGLVNEISHEMLVAINGLDFGDALTKLMSKNICNYSVRLLRDTTGLDNRTISNMEKGNNLTKINVISACLGIQIPFRVSNKMLQLAELSLNFDLPGKKGEENGIYDSVLHLKWATDYEDVYEELKEQNYEYLLHRPKKIK

SEQ ID NO: 2 - DNA, 6701 bp, Artificial
  Artificial gene
gcggccgctcagtcacctcctagctgactcaaatcaatgcgtgtttcataaagaccagtgatggattgatggataagagtggcatctaaaacttcttttgtagacgtatatcgtttacgatcaattgttgtatcaaaatatttaaaagcagcgggagctccaagattcgtcaacgtaaataaatgaataatattttctgcttgttcacgtattggtttgtctctatgtttgttatatgcactaagaactttatctaaattggcatctgctaaaataacacgcttagaaaattcactgatttgctcaataatctcatctaaataatgcttatgctgctccacaaacaattgtttttgttcgttatcttctggactacccttcaacttttcataatgactagctaaatataaaaaattcacatatttgcttggcagagccagctcatttcctttttgtaattctccggcactagccagcatccgtttacgaccgttttctaactcaaaaagactatatttaggtagtttaatgattaagtcttttttaacttccttatatcctttagcttctaaaaagtcaatcggatttttttcaaaggaacttctttccataattgtgatccctagtaactctttaacggattttaacttcttcgatttccctttttccaccttagcaaccactaggactgaataagctaccgttggactatcaaaaccaccatatttttttggatcccagtcttttttacgagcaataagcttgtccgaatttctttttggtaaaattgactccttggagaatccgcctgtctgtacttctgttttcttgacaatattgacttggggcatggacaatactttgcgcactgtggcaaaatctcgccctttatcccagacaatttctccagtttccccattagtttcgattagagggcgtttgcgaatctctccatttgcaagtgtaatttctgttttgaagaagttcatgatattagagtaaaagaaatattttgcggttgctttgcctatttcttgctcagacttagcaatcattttacgaacatcataaactttataatcaccatagacaaactccgattcaagttttggatatttcttaatcaaagcagttccaacgacggcatttagatacgcatcatgggcatgatggtaattgttaatctcacgtactttatagaattggaaatcttttcggaagtcagaaactaatttagattttaaggtaatcactttaacctctcgaataagtttatcattttcatcgtatttagtattcatgcgactatccaaaatttgtgccacatgcttagtgatttggcgagtttcaaccaattggcgtttgataaaaccagctttatcaagttcactcaaacctccacgttcagctttcgttaaattatcaaacttacgttgagtgattaacttggcgtttagaagttgtctccaatagtttttcatctttttgactacttcttcacttggaacgttatccgatttaccacgatttttatcagaacgcgttaagaccttattgtctattgaatcgtctttaaggaaactttgtggaacaatgtgatcgacatcataatcacttaaacgattaatatctaattcttggtccacatacatgtctcttccattttggagataatagagatagagcttttcattttgcaattgagtattttcaacaggatgctctttaagaatctgacttcctaattctttgataccttcttcgattcgtttcatacgctctcgcgaatttttctggcccttttgagttgtctgattttcacgtgccatttcaataacgatattttctggcttatgccgccccattactttgaccaattcatcaacaacttttacagtctgtaaaataccttttttaatagcagggctaccagctaaatttgcaatatgttcatgtaaactatcgccttgtccagacacttgtgctttttgaatgtcttctttaaatgtcaaactatcatcatggatcagctgcataaaattgcgattggcaaaaccatctgatttcaaaaaatctaatattgttttgccagattgcttatccctaataccattaatcaattttcgagacaaacgtccccaaccagtataacggcgacgtttaagctgtttcatcaccttatcatcaaagaggtgagcatatgttttaagtctttcctcaatcatctccctatcttcaaataaggtcaatgttaaaacaatatcctctaagatatcttcattttcttcattatccaaaaaatctttatctttaataatttttagcaaatcatggtaggtacctaatgaagcattaaatctatcttcaactcctgaaatttcaacactatcaaaacattctatttttttgaaataatcttcttttaattgcttaacggttacttttcgatttgttttgaagagtaaatcaacaatggctttcttctgttcacctgaaagaaatgctggttttcgcattccttcagtaacatatttgacctttgtcaattcgttataaaccgtaaaatactcataaagcaaactatgttttggtagtactttttcatttggaagatttttatcaaagtttgtcatgcgttcaataaatgattgagctgaagcacctttatcgacaacttcttcaaaattccatggggtaattgtttcttcagacttccgagtcatccatgcaaaacgactattgccacgcgccaatggaccaacataataaggaattcgaaaagtcaagattttttcaatcttctcacgattgtcttttaaaaatggataaaagtcttcttgtcttctcaaaatagcatgcagctcacccaagtgaatttgatggggaatagagccgttgtcaaaggtccgttgcttgcgcagcaaatcttcacgatttagtttcaccaataattcctcagtaccatccattttttctaaaattggtttgataaatttataaaattcttcttggctagctcccccatcaatataacctgcatatccgttttttgattgatcaaaaaagatttctttatacttttctggaagttgttgtcgaactaaagcttttaaaagagtcaagtcttgatgatgttcatcgtagcgtttaatcattgaagctgataggggagccttagttatttcagtatttactcttaggatatctgaaagtaaaatagcatctgataaattcttagctgccaaaaacaaatcagcatattgatctccaatttgcgccaataaattatctaaatcatcatcgtaagtatcttttgaaagctgtaatttagcatcttctgccaaatcaaaatttgatttaaaattaggggtcaaacccaatgacaaagcaatgagattcccaaataagccatttttcttctcaccggggagctgagcaatgagattttctaatcgtcttgatttactcaatcgtgcagaaagaatcgctttagcatctactccacttgcgttaatagggttttcttcaaataattgattgtaggtttgtaccaactggataaatagtttgtccacatcactattatcaggatttaaatctccctcaatcaaaaaatgaccacgaaacttaatcatatgcgctaaggccaaatagattaagcgcaaatccgctttatcagtagaatctaccaatttttttcgcagatgatagatagttggatatttctcatgataagcaacttcatctactatatttccaaaaataggatgacgttcatgcttcttgtcttcttccaccaaaaaagactcttcaagtcgatgaaagaaactatcatctactttcgccatctcatttgaaaaaatctcctgtagataacaaatacgattcttccgacgtgtataccttctacgagctgtccgtttgagacgagtcgcttccgctgtctctccactgtcaaataaaagagcccctataagattttttttgatactgtggcggtctgtatttcccagaaccttgaactttttagacggaaccttataatcatcagtgatcaccgcccatccgacgctatttgtgccgatatctaagcctattgagtatttcttatccattctaactaacctcctaaattttgatacggggtaacagataaaccatttcaatctatttcataagttccatagtttatccctaatttatacgttttctctaacaacttaattatacccactattattatttttatcaatatattttgttaaaaagtcgactttatatttagtcccttgccttgcctacaagggatttcctattcctttcatttacaattcatacgtataaaatccaaatttttcttgacatttatacacataaatattatgatttatataggtaatcgctttcataaaatatattacccttaggaaatcaaatgattataagtcatatatgaaaacgttatatataattgatatgtttacatttgtaacttagatttctctttgatttccacatatataaatcttaaggaggagttttcccaagcattgataatgaagggttttagagctagaaatagcaagttaaaataaggctagtccgttatcaacttgaaaaagtggcaccgagtcggtgctttttttataaaaataagaagcctgcatttgcaggcttcttatttttatgcggaatggagatatcgtaatagctgatgctgcagaagatgaaactgtcgggaaatgcagtgaaatcaaaggaataggttgtataagtattgtctctggattgcatacgataccttgtagacctatcaagacatttgagactggttaccttggatattatatgaattcaagcgcttatcatgatcagttattaccactgatacagggtacaaaaatttcatctatttcaaagtcggcactacagaacacggaaataatttacccagattcagaaaaggaacaactaaaaattgggcagttttttcaaaacctggatagccttatcactcttcatcagcgaaagtatgataagctaattattgttaaaaagtctatgttggaaaaaatgttccctatagatggtagcggtagcaatgtgcctgaaattcgatttggtggttttactgatgattggaaatttcgtaagttgggagactgcttttcagaaaggtctgaaagtatgcctgatggggaattgatttctgttactataaatgatggaataaagaaattttcagaacttggaagacatgatacttctaacgatgataaatctaagtataagaaggtgtgtgttggagacatagcctataactctatgagaatgtggcaaggtgcaagtggatactccccatacgaaggtatagttagtcctgcatatacagtacttgccccaaataatggaattgactcaaaatgtatttcatatttatttaaacgccctgatatgattcatacattccaagttaattcacaaggaataacatctgataactggaatttgaaatatcaggcattgagtgaaattgagattttgattccaaatgatattcaagaacaaaaatatattgctgaatacttcactggtctcgacaacctgatcactcttcatcagcgtaaactcgaaaaattaagaaatataaggttttcttgtacggaaaaaatgtttgtttaggggggctgagattgtaatgtttagatttagaccagattaacgtctggtctatttttttgctcaaaaaaattttttgttgaactttgttcaacgaaatctctcactaatttggagaatttttaaatgcagaattaaatatgtgcgttattaaggctttttggatgttgaactttattcaacgggcaaaaaagctagaaagtataaaatgaatttatagtaaaaatcaatatcacaggcctgattagctataagggcatgggatacaaatattggtccatcacagctacagcgtgatgggtgcaatagaagtacccttatttccttatgctatttttcaggcgatgggtcggtgtacttcaaaaggcaccgactctatttgtttctcttgtccttctgcaagaaccaggcagaaaggcaggaaactttatgagaaactttaaaaccagcaaaaagaacagaaccaattacatttattattcagctgatggacaggcaataaaaattaaacctaatgaggatgaagctacaactactatcattgtcactttacatagctgggatgatgcagaatttaatgctgataaacgcgaaagataccatgttccagtatacatggatgcttattgtaatgaaaacgaagatgttgcagctgatcgcaatccatatttagttgatactgcttctgatccactggaaagtattattcaatccattgaggaaacagagcataaagaaaagataggcaggctaagagctgcaattggaactttaaagccacagcagaaagagcttataaaaaaagtattctttgaaaaacgcactaatgtaagtattgctgatgaagaaggtgttacagaagcagctattagaaatcggctaaaaaagatttatgagaaacttcgcaaaaaaatctaaaaataggggcacgaatccccctaaataaagtttcaaaaaagggggttcgattccctaaagttttttgcatatggacagaggggtaaataaaacccctcctcgag

SEQ ID NO: 3 - DNA, 43 bp, Artificial
  Synthetic DNA
gatccgcggccgctctagagtcgacgggcccctcgagcctcga

SEQ ID NO: 4 - DNA, 13255 bp, Artificial
  Vector pCL2-TypeIKO
gaattcgagctcggtacccggggatccgcggccgctcagtcacctcctagctgactcaaatcaatgcgtgtttcataaagaccagtgatggattgatggataagagtggcatctaaaacttcttttgtagacgtatatcgtttacgatcaattgttgtatcaaaatatttaaaagcagcgggagctccaagattcgtcaacgtaaataaatgaataatattttctgcttgttcacgtattggtttgtctctatgtttgttatatgcactaagaactttatctaaattggcatctgctaaaataacacgcttagaaaattcactgatttgctcaataatctcatctaaataatgcttatgctgctccacaaacaattgtttttgttcgttatcttctggactacccttcaacttttcataatgactagctaaatataaaaaattcacatatttgcttggcagagccagctcatttcctttttgtaattctccggcactagccagcatccgtttacgaccgttttctaactcaaaaagactatatttaggtagtttaatgattaagtcttttttaacttccttatatcctttagcttctaaaaagtcaatcggatttttttcaaaggaacttctttccataattgtgatccctagtaactctttaacggattttaacttcttcgatttccctttttccaccttagcaaccactaggactgaataagctaccgttggactatcaaaaccaccatatttttttggatcccagtcttttttacgagcaataagcttgtccgaatttctttttggtaaaattgactccttggagaatccgcctgtctgtacttctgttttcttgacaatattgacttggggcatggacaatactttgcgcactgtggcaaaatctcgccctttatcccagacaatttctccagtttccccattagtttcgattagagggcgtttgcgaatctctccatttgcaagtgtaatttctgttttgaagaagttcatgatattagagtaaaagaaatattttgcggttgctttgcctatttcttgctcagacttagcaatcattttacgaacatcataaactttataatcaccatagacaaactccgattcaagttttggatatttcttaatcaaagcagttccaacgacggcatttagatacgcatcatgggcatgatggtaattgttaatctcacgtactttatagaattggaaatcttttcggaagtcagaaactaatttagattttaaggtaatcactttaacctctcgaataagtttatcattttcatcgtatttagtattcatgcgactatccaaaatttgtgccacatgcttagtgatttggcgagtttcaaccaattggcgtttgataaaaccagctttatcaagttcactcaaacctccacgttcagctttcgttaaattatcaaacttacgttgagtgattaacttggcgtttagaagttgtctccaatagtttttcatctttttgactacttcttcacttggaacgttatccgatttaccacgatttttatcagaacgcgttaagaccttattgtctattgaatcgtctttaaggaaactttgtggaacaatgtgatcgacatcataatcacttaaacgattaatatctaattcttggtccacatacatgtctcttccattttggagataatagagatagagcttttcattttgcaattgagtattttcaacaggatgctctttaagaatctgacttcctaattctttgataccttcttcgattcgtttcatacgctctcgcgaatttttctggcccttttgagttgtctgattttcacgtgccatttcaataacgatattttctggcttatgccgccccattactttgaccaattcatcaacaacttttacagtctgtaaaataccttttttaatagcagggctaccagctaaatttgcaatatgttcatgtaaactatcgccttgtccagacacttgtgctttttgaatgtcttctttaaatgtcaaactatcatcatggatcagctgcataaaattgcgattggcaaaaccatctgatttcaaaaaatctaatattgttttgccagattgcttatccctaataccattaatcaattttcgagacaaacgtccccaaccagtataacggcgacgtttaagctgtttcatcaccttatcatcaaagaggtgagcatatgttttaagtctttcctcaatcatctccctatcttcaaataaggtcaatgttaaaacaatatcctctaagatatcttcattttcttcattatccaaaaaatctttatctttaataatttttagcaaatcatggtaggtacctaatgaagcattaaatctatcttcaactcctgaaatttcaacactatcaaaacattctatttttttgaaataatcttcttttaattgcttaacggttacttttcgatttgttttgaagagtaaatcaacaatggctttcttctgttcacctgaaagaaatgctggttttcgcattccttcagtaacatatttgacctttgtcaattcgttataaaccgtaaaatactcataaagcaaactatgttttggtagtactttttcatttggaagatttttatcaaagtttgtcatgcgttcaataaatgattgagctgaagcacctttatcgacaacttcttcaaaattccatggggtaattgtttcttcagacttccgagtcatccatgcaaaacgactattgccacgcgccaatggaccaacataataaggaattcgaaaagtcaagattttttcaatcttctcacgattgtcttttaaaaatggataaaagtcttcttgtcttctcaaaatagcatgcagctcacccaagtgaatttgatggggaatagagccgttgtcaaaggtccgttgcttgcgcagcaaatcttcacgatttagtttcaccaataattcctcagtaccatccattttttctaaaattggtttgataaatttataaaattcttcttggctagctcccccatcaatataacctgcatatccgttttttgattgatcaaaaaagatttctttatacttttctggaagttgttgtcgaactaaagcttttaaaagagtcaagtcttgatgatgttcatcgtagcgtttaatcattgaagctgataggggagccttagttatttcagtatttactcttaggatatctgaaagtaaaatagcatctgataaattcttagctgccaaaaacaaatcagcatattgatctccaatttgcgccaataaattatctaaatcatcatcgtaagtatcttttgaaagctgtaatttagcatcttctgccaaatcaaaatttgatttaaaattaggggtcaaacccaatgacaaagcaatgagattcccaaataagccatttttcttctcaccggggagctgagcaatgagattttctaatcgtcttgatttactcaatcgtgcagaaagaatcgctttagcatctactccacttgcgttaatagggttttcttcaaataattgattgtaggtttgtaccaactggataaatagtttgtccacatcactattatcaggatttaaatctccctcaatcaaaaaatgaccacgaaacttaatcatatgcgctaaggccaaatagattaagcgcaaatccgctttatcagtagaatctaccaatttttttcgcagatgatagatagttggatatttctcatgataagcaacttcatctactatatttccaaaaataggatgacgttcatgcttcttgtcttcttccaccaaaaaagactcttcaagtcgatgaaagaaactatcatctactttcgccatctcatttgaaaaaatctcctgtagataacaaatacgattcttccgacgtgtataccttctacgagctgtccgtttgagacgagtcgcttccgctgtctctccactgtcaaataaaagagcccctataagattttttttgatactgtggcggtctgtatttcccagaaccttgaactttttagacggaaccttataatcatcagtgatcaccgcccatccgacgctatttgtgccgatatctaagcctattgagtatttcttatccattctaactaacctcctaaattttgatacggggtaacagataaaccatttcaatctatttcataagttccatagtttatccctaatttatacgttttctctaacaacttaattatacccactattattatttttatcaatatattttgttaaaaagtcgactttatatttagtcccttgccttgcctacaagggatttcctattcctttcatttacaattcatacgtataaaatccaaatttttcttgacatttatacacataaatattatgatttatataggtaatcgctttcataaaatatattacccttaggaaatcaaatgattataagtcatatatgaaaacgttatatataattgatatgtttacatttgtaacttagatttctctttgatttccacatatataaatcttaaggaggagttttcccaagcattgataatgaagggttttagagctagaaatagcaagttaaaataaggctagtccgttatcaacttgaaaaagtggcaccgagtcggtgctttttttataaaaataagaagcctgcatttgcaggcttcttatttttatgcggaatggagatatcgtaatagctgatgctgcagaagatgaaactgtcgggaaatgcagtgaaatcaaaggaataggttgtataagtattgtctctggattgcatacgataccttgtagacctatcaagacatttgagactggttaccttggatattatatgaattcaagcgcttatcatgatcagttattaccactgatacagggtacaaaaatttcatctatttcaaagtcggcactacagaacacggaaataatttacccagattcagaaaaggaacaactaaaaattgggcagttttttcaaaacctggatagccttatcactcttcatcagcgaaagtatgataagctaattattgttaaaaagtctatgttggaaaaaatgttccctatagatggtagcggtagcaatgtgcctgaaattcgatttggtggttttactgatgattggaaatttcgtaagttgggagactgcttttcagaaaggtctgaaagtatgcctgatggggaattgatttctgttactataaatgatggaataaagaaattttcagaacttggaagacatgatacttctaacgatgataaatctaagtataagaaggtgtgtgttggagacatagcctataactctatgagaatgtggcaaggtgcaagtggatactccccatacgaaggtatagttagtcctgcatatacagtacttgccccaaataatggaattgactcaaaatgtatttcatatttatttaaacgccctgatatgattcatacattccaagttaattcacaaggaataacatctgataactggaatttgaaatatcaggcattgagtgaaattgagattttgattccaaatgatattcaagaacaaaaatatattgctgaatacttcactggtctcgacaacctgatcactcttcatcagcgtaaactcgaaaaattaagaaatataaggttttcttgtacggaaaaaatgtttgtttaggggggctgagattgtaatgtttagatttagaccagattaacgtctggtctatttttttgctcaaaaaaattttttgttgaactttgttcaacgaaatctctcactaatttggagaatttttaaatgcagaattaaatatgtgcgttattaaggctttttggatgttgaactttattcaacgggcaaaaaagctagaaagtataaaatgaatttatagtaaaaatcaatatcacaggcctgattagctataagggcatgggatacaaatattggtccatcacagctacagcgtgatgggtgcaatagaagtacccttatttccttatgctatttttcaggcgatgggtcggtgtacttcaaaaggcaccgactctatttgtttctcttgtccttctgcaagaaccaggcagaaaggcaggaaactttatgagaaactttaaaaccagcaaaaagaacagaaccaattacatttattattcagctgatggacaggcaataaaaattaaacctaatgaggatgaagctacaactactatcattgtcactttacatagctgggatgatgcagaatttaatgctgataaacgcgaaagataccatgttccagtatacatggatgcttattgtaatgaaaacgaagatgttgcagctgatcgcaatccatatttagttgatactgcttctgatccactggaaagtattattcaatccattgaggaaacagagcataaagaaaagataggcaggctaagagctgcaattggaactttaaagccacagcagaaagagcttataaaaaaagtattctttgaaaaacgcactaatgtaagtattgctgatgaagaaggtgttacagaagcagctattagaaatcggctaaaaaagatttatgagaaacttcgcaaaaaaatctaaaaataggggcacgaatccccctaaataaagtttcaaaaaagggggttcgattccctaaagttttttgcatatggacagaggggtaaataaaacccctcctcgagccctgcaggcatgcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtaagtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccgggatcgatccccgccgagcgcttagtgggaatttgtaccccttatcgatacaaattccccgtaggcgctagggacactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 5 - DNA, 6038 bp, Artificial
  Vector pUC-pta-ermC
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctttgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccgtgcagaaggataggacataccctgtgacattttttcctttaaaaataatttaaattgggtaggctcttctgcaagaatttttgcaatagatttcagcaagtttatattactatattcgcttccaaaacaaagattttttactacacccaagttttctaagagacttacagcaccataggcaaaaaattcagcagaagatagactgtagataacaggaagttcaaataccaggtctactccatttagaagtgccattttggttttagtccatttgtcaactatagatggtgaacctctttgcacgaagttaccactcataactgctattacagcatcacattttgtagcagaacgagcactttcaatatgatatttatgtccattgtgaaagggattatattcaactattattccagttacgttcatagaaattttcctttctaaaatattttattccatgtcaagaactctgtttatttcattaaagaactataagtacaaagtatagggcatttgaaaaaataggctagtatattgattgattatttattttaaaatgcctaagtgaaatatatacatattataacaataaaataagtattagtgtaggatttttaaatagagtatctattttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgaaattgatggaaaaaatttggagtaaggcaaaggaagacaaaaaaaagattgtcttagctgaaggagaagaagaaagaactcttcaagcttgtgaaaaaataattaaagagggtattgcaaatttaatccttgtagggaatgaaaaggtaataaaagaaaaagcgtcaaaattaggtgtaagtttaaatggagcagaaatagtagatccagagacttcagataaactaaaggcatatgcagatgctttttatgaattgagaaagaagaagggaataacgccagaaaaagcggatcttcggaataggaacttccgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgattatgtcttttgcgcattcacttcttttctatataaatatgagcgaagcgaataagcgtcggaaaagcagcaaaaagtttcctttttgctgttggagcatgggggttcagggggtgcagtatcgaagttcctggaatggatccaaaagtagcaatgctttcattttctactaagggaagtgcaaaacacgaattagtagacaaagttagaaatgctgtagagattgcaaaaaaagctaaaccagatttaagtttagacggagaattacaattagatgcctctatcgtagaaaaggttgcaagtttaaaggctcctggaagtgaagtagcaggaaaagcaaatgtacttgtatttccagatctccaagcaggaaatataggctataaactcgttcaaagatttgcaaaagcagatgctataggacctgtatgccaaggatttgcaaaacctataaatgatttgtcaagaggatgtaattctgatgatatagtaaatgtagtagctgtaacagcagttcaagcacaagctcaaaagtaataacaaaaagcataaatgattcatttttaggaggaatattaaacatgaaaatattagtagtaaactgtggaagttcatctttaaaatatcaacttattgatatgcaagatgaaagtgttgtagcaaagggtcttgtagaaagaataggaatggacggttcaattttaacacacaaagttaatggagaaaagtttgttacagagcaaacaatggaagaccacaaagttgctatacaattagtattaaatgctcttgtagataaaaaacatggtgtaataaaagacatgtcagaaatatccgctgtaggacatagagtcttgcacggtggaaagaaatatgcagcatccattcttattgacgaaaatgtaatgaaagcaatagaagaatgtatcccactaggaccactacataatccagctaatataatgggaatagatgcttgtaaaaaattaatgccaaatactccaatggtagcagtatttgatacagcatttcatcagacaatgccagattatgcttatacttatgcaataccttatgatatatctgaaaagtatgatatcagaaaatatggttttcatggaacttctcatagattcgtttcaattgaagcagctaaattattaaagaaagatccaaaagatcttaagttaataacttgtcatttaggaaatggagctagcatatgtgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

(19) United States
(12) Patent Application Publication
(10) Pub. No.: US 2019/0233854 A1
(43) Pub. Date: Aug. 1, 2019

(54) OBLIGATELY ANAEROBIC ACETIC ACID-PRODUCING MICROORGANISM, AND RECOMBINANT MICROORGANISM

(71) Applicant: SEKISUI CHEMICAL CO., LTD., Osaka (JP)

(72) Inventors: Kana MATSUSHIMA, Nagareyama-shi, Chiba (JP); Masahiro FURUTANI, Arakawa-ku, Tokyo (JP); Kazufumi KAWABATA, Tsukuba-shi, Ibaraki (JP)

(73) Assignee: SEKISUI CHEMICAL CO., LTD., Osaka (JP)

(21) Appl. No.: 16/335,377

(22) PCT Filed: Sep. 5, 2017
(86) PCT No.: PCT/JP2017/031922

§ 371 Date: Mar. 21, 2019

(30) Foreign Application Priority Data
Oct. 13, 2016 (JP) 2016-201947

(51) Int. Cl. C12P 7/54; C12N 1/20; C12R 1/145

(52) U.S. Cl. CPC C12P 7/54; C12Y 102/99002; C12R 1/145; C12N 1/20

(57) ABSTRACT

Provided is a modified obligately anaerobic acetogen in which activity of at least one enzyme selected from the group consisting of a restriction enzyme, a modification enzyme, and a recognition enzyme constituting a type I restriction modification system enzyme is deleted or suppressed. Preferably, the activity of at least the restriction enzyme is deleted or suppressed. The obligately anaerobic acetogen is preferably a Clostridium bacterium or a Moorella bacterium, and particularly preferably Clostridium ljungdahlii.

### TECHNICAL FIELD

The present invention relates to an obligately anaerobic acetogen, and a recombinant microorganism. The obligately anaerobic acetogen of the present invention has high transformation efficiency by a foreign gene.

### BACKGROUND ART

Obligately anaerobic acetogens have conventionally been used for fermentation production of butanol, acetone, and the like. In particular, a Clostridium bacterium is used for production of bioethanol and production of organic acid such as acetic acid and lactic acid, using a lignocellulose-based biomass or syngas (synthesis gas). A Clostridium bacterium is expected to be used in the field of medicine.

Recombinant microorganisms using obligately anaerobic acetogen such as a Clostridium bacterium as a host are useful to improve the production ability of a useful substance. However, in general, since the transformation efficiency of a Clostridium bacterium is very low, if is difficult to obtain desired recombinant microorganisms. This is a major impediment to development in the art.

In general, microorganisms have developed a restriction modification system to protect themselves from foreign DNA such as viruses and phages. The restriction modification system includes a restriction enzyme that cleaves DNA and a modification enzyme that methylates DNA. By the action of the modification enzyme, DNA that has been modified by methylation is protected from cleavage by restriction enzymes, and genomic DNA of the microorganism itself is not cleaved. The microorganism distinguishes the DNA of its own from a foreign DNA such as viruses, by the methylation modification, and protects itself by cleavage of the foreign DNA by restriction enzymes.

The restriction modification systems are roughly classified into four types, that is, type I, type II, type III, and type VI depending on a structure of subunit, a cleavage site, and characteristics of a cofactor. Restriction enzymes of type I, type II and type III restriction modification systems distinguish and cleave DNA that has not been methylated (see, for example, Non-Patent Document 1). However, when DNA is recognized and modified by a methylase that is a pair of the restriction enzyme, the restriction enzyme cannot cleave the DNA. On the other hand, the type IV restriction modification system includes only a restriction enzyme, and is a methylation-dependent restriction enzyme that distinguishes and cleaves DNA having methylation form of a foreign DNA. In this way, the restriction modification system is a strong system that protects microorganism cells from foreign DNA. Therefore, when foreign DNA is introduced into a microorganism to form a recombinant, it is important to overcome the restriction modification system of a host.

In bacteria, improvement of transformation efficiency using a restriction modification system, in particular, a type II restriction modification enzyme (methyltransferase) has been studied, and described in, for example, Patent Document 1. However, its implementation includes complex operations, for example, it needs a plurality of steps, and needs all methyltransferases of a host, so that it has many problems to be solved.

Furthermore, there has been no report that transformation efficiency is improved by modification (alteration) of the type I restriction modification system, in particular, by deletion of the type I restriction enzyme.

Incidentally, among the Clostridium bacteria, in particular, Clostridium ljungdahlii is much expected. Clostridium ljungdahlii has advantages in that the genome information is publicly available, culture is easy, and synthesis gas (syngas) can be assimilated.

The syngas is a mixed gas mainly including carbon monoxide, carbon dioxide and hydrogen, which is efficiently obtained from waste, natural gas and coal by the action of a metal catalyst at high temperature and pressure. The syngas including carbon monoxide, carbon dioxide, and hydrogen is produced and available almost permanently as syngas derived from waste or industrial waste gas, natural gas, or syngas derived from coal. Thus, a permanent production method of a substance using microorganisms having syngas assimilating ability has been expected. By using the recombinant microorganism, the productivity of a target substance can be expected to be improved. However, there are very few reports as to the use of the syngas assimilating microorganism as a recombinant. This is thought to be mainly because as mentioned above, the transformation efficiency in a Clostridium bacterium is low, so that the desired recombinant cannot be easily obtained.

### PRIOR ART DOCUMENTS

### Patent Documents

Patent Document 1: JP 2015-515268 A

### Non-Patent Documents

Non-Patent Document 1: Wil A. M. Loenen et al., Nucleic Acids Research, 2014, vol. 42(1), p. 22-44

### DISCLOSURE OF INVENTION

### Technical Problem

In view of the above-mentioned circumstances, the present invention has an object to provide an obligately anaerobic acetogen, such as a Clostridium bacterium whose transformation efficiency is improved.

### Solution to Problem

One aspect of the present invention to solve the above-mentioned problem is a modified obligately anaerobic acetogen in which activity of at least one enzyme selected from the group consisting of a restriction enzyme, a modification enzyme, and a recognition enzyme constituting a type I restriction modification system enzyme is deleted or suppressed.

This aspect relates to a modified obligately anaerobic acetogen. In the obligately anaerobic acetogen of this aspect, the activity of at least one enzyme selected from the group consisting of a restriction enzyme, a modification enzyme, and a recognition enzyme constituting a type I restriction modification system enzyme is deleted or suppressed. Therefore, the function of the type I restriction modification system is lost or reduced. Since the obligately anaerobic acetogen of this aspect is tolerant of degradation (cleavage property) to foreign DNA, when it is used as a host, the transformation efficiency is high. When the obligately anaerobic acetogen of this aspect is used, a recombinant of the obligately anaerobic acetogen can be obtained at high efficiency.

Preferably, the activity of at least the restriction enzyme is deleted or suppressed.

Preferably, a part or whole of a gene encoding at least one enzyme selected from the group consisting of the restriction enzyme, the modification enzyme, and the recognition enzyme is deleted.

Preferably, a part or whole of a gene encoding at least the restriction enzyme is deleted.

Preferably, the activity of the restriction enzyme, the modification enzyme, or the recognition enzyme is deleted

Preferably, the obligately anaerobic acetogen is capable of proliferating using carbon monoxide or carbon dioxide as a sole carbon source.

Preferably, the obligately anaerobic acetogen includes carbon monoxide dehydrogenase.

Preferably, the obligately anaerobic acetogen has a function of synthesizing acetyl-CoA from methyl tetrahydrofolate, carbon monoxide, and CoA.

Preferably, the obligately anaerobic acetogen is a bacterium.

Preferably, the obligately anaerobic acetogen is a Clostridium bacterium or a Moorella bacterium.

Preferably, the obligately anaerobic acetogen is Clostridium ljungdahlii.

Preferably, the restriction enzyme is a protein including an amino acid sequence of SEQ ID NO: 1.

Preferably, the obligately anaerobic acetogen is Clostridium ljungdahlii, the activity of at least the restriction enzyme is deleted or suppressed, and a part or whole of a gene encoding at least the restriction enzyme is deleted.

Another aspect of the present invention is a recombinant microorganism including the above-described obligately anaerobic acetogen into which a foreign gene encoding a target protein is introduced, and being capable of expressing the target protein.

This aspect relates to a recombinant microorganism. In the recombinant microorganism of this aspect, a foreign gene encoding a target protein is introduced into the above-mentioned obligately anaerobic acetogen as a host, and the target protein can be expressed. Production of the recombinant microorganism of this aspect is easy.

Preferably, the foreign gene is incorporated into a genome.

### EFFECT OF INVENTION

With the obligately anaerobic acetogen of the present invention, a recombinant of an obligately anaerobic acetogen can be obtained with high efficiency.

The recombinant microorganism of the present invention can be easily produced.

### BRIEF DESCRIPTION OF DRAWINGS

FIG. 1 is an explanatory diagram showing a configuration of a pCL2-TypeIKO vector.

### BEST MODE FOR CARRYING OUT THE INVENTION

Hereinafter, the exemplary embodiment of the present invention will be described. Note here that in the present invention, all the terms “gene” can be replaced with terms “nucleic acid” or “DNA”.

In the modified obligately anaerobic acetogen of the present invention, activity of at least one enzyme selected from the group consisting of a restriction enzyme, a modification enzyme, and a recognition enzyme constituting a type I restriction modification system enzyme is deleted or suppressed. Herein, obligatory anaerobic property means a property which makes growth in the presence of oxygen virtually impossible and is the same meaning as strictly anaerobic property. Acetogen means a microorganism that produces acetate by anaerobic respiration.

In general, the type I restriction modification system enzyme functions as a complex consisting of three enzyme subunits, recognition, modification, and restriction (cleavage). The recognition enzyme (type I recognition enzyme) recognizes a DNA sequence that is specific to a species or specific to each enzyme. The modification enzyme (type I modification enzyme) carries out methylation by methyltransferase activity. The restriction enzyme (type I restriction enzyme) carries out cleavage of DNA by endonuclease activity.

It is known that when activity of at least one of these three enzymes is lost, the type I restriction modification system does not function (see, Non-Patent Document 1 mentioned above). In the obligately anaerobic acetogen of the present invention, activity of at least one enzyme selected from the group consisting of a restriction enzyme, a modification enzyme, and a recognition enzyme constituting the type I restriction modification system enzyme is deleted or suppressed. In other words, the function of the type I restriction modification system is lost or reduced.

Deletion of the enzyme activity means that activity of the enzyme is absent. Suppression of the enzyme activity means that as compared with the enzyme activity of a microorganism before modification or alteration (i.e. wild type), the activity is reduced to 50% or less, preferably 25% or less, and more preferably 10% or less. Measurement of the activity of the type I restriction modification system enzyme can be carried out by, for example, a method (DNA cleavage assay) described in Piero R. Bianco et al., Nucleic Acids Research, 2009, Vol. 37, No. 10, 3377-3390.

Examples of techniques for deleting or suppressing the activity of enzyme include roughly two techniques, a technique of reducing (including reducing to zero) the number of enzyme molecules to be expressed and a technique of reducing (including reducing to zero) the activity of the enzyme itself to be expressed.

Examples of the technique of reducing the number of enzyme molecules to be expressed include reducing the expression amount of the gene of the type I restriction modification system enzyme at a transcription level or a translation level. Specific examples of the technique include a method of modifying (altering) an expression regulatory sequence such as a promoter sequence and a Shine-Dalgarno (SD) sequence of the gene of the type I restriction modification system enzyme to reduce the expression amount of the gene.

Examples of the technique of reducing the activity of enzyme itself to be expressed include deleting a part or whole of the gene of the type I restriction modification system enzyme on a genome. Examples thereof include an embodiment in which some or all of the genes encoding the restriction enzyme, the modification enzyme, or the recognition enzyme are deleted. Among them, preferred is an embodiment in which some or all of the genes encoding at least a restriction enzyme are deleted.

Other techniques for reducing the activity of the enzyme itself to be expressed include introduction of mutation into a gene of the type I restriction modification system enzyme on the genome. For example, an objective microorganism is subjected to treatment with a mutagenic agent and the like so as to introduce mutation into a gene of the target type I restriction modification system enzyme. For example, objective microorganisms are treated by radiation irradiation or with mutagenic agents such as N-methyl-N′-nitro-N-nitroso guanidine (NTG) and nitrous acid, and then a mutant microorganism (modified microorganism) in which the activity of the type I restriction modification system enzyme is deleted or suppressed can be obtained.

The enzyme gene originally possessed by the host microorganism may be substituted with another gene having lower enzyme activity. The activity of the type I restriction modification system enzyme itself can be reduced by replacing, for example, an existing gene of the type I restriction modification system enzyme on the genome with a mutant gene that caused a frame shift in the gene, a mutant gene in which a point mutation is introduced in the gene, and a partial fragment gene of the gene.

Substitution of a gene can be carried out by a genome editing technique (CRISPR-Cas9 system). In the genome editing technique, any DNA sequences can be targeted site-specifically under the conditions in which Cas9 as endonuclease or mutant Cas9 that does not have catalyst activity form a complex with sgRNA (single guide RNA), in a vicinity of a region in which sgRNA and the target DNA are complementary bonded, and a specific short nucleotide sequence called PAM (proto-spacer adjacent motif) exists. Use of the genome editing technique in the syngas assimilating microorganism is described in, for example, “Huang H et al., ACS Synth Biol. 2016 Jun. 15.”

The substitution of the gene can also be carried out by homologous recombination. The sequence identity of nucleotide sequence required for the homologous recombination is preferably 70% or more, more preferably 80% or more, further preferably 90% or more, and particularly preferably 95% or more. Note here that gene manipulation methods by the homologous recombination have been established for many bacteria and the like, and the methods include a method using a linear DNA, and a method using a plasmid (see U.S. Pat. No. 6,303,383 B1, JP H05-007491 A, etc.). Technique of homologous recombination by plasmid in a Clostridium bacterium is described in, for example, “Heap J T et al., J. Microbiol. Methods, 2007, vol. 70 (3), p. 452-64”.

Together with a gene encoding Cas9 or a gene encoding sgRNA, a homologous DNA sequence for deleting a part or whole of the type I restriction modification enzyme may be introduced.

Some or all of the type I restriction modification system enzyme genes can be deleted by introducing a DNA sequence (DNA fragment) such as a foreign gene into an unmodified microorganism. For example, some or all of the type I restriction modification system enzyme gene can be deleted by inserting a DNA sequence into the structural gene of the type I restriction modification system enzyme. The DNA sequence to be introduced at this time is not particularly limited, and may be a DNA sequence originally possessed by the microorganism, or may be a heterologous DNA sequence that is not originally possessed by the microorganism. Examples of the DNA sequence to be introduced include an antibiotics resistant gene.

A method for introducing a DNA fragment into a microorganism is not particularly limited, and it may be appropriately selected depending on types of microorganisms. For example, it is possible to use a vector that can be introduced into a microorganism and that cannot autonomously replicate after recombination. For example, when a microorganism is a prokaryote such as a bacterium, it is possible to use a vector that can be incorporated into chromosome (genome) in a microorganism. For example, preferably, the above-mentioned DNA fragment is introduced using a vector, and then the vector is eliminated.

Examples of the type I restriction modification system enzyme of the obligately anaerobic acetogen include the followings.

Examples of type I restriction enzyme (for example, EC: 3.1.21.3): ADK13415 (Clostridium ljungdahlii-hsdR); AKN29959 (Clostridium carboxidivorans-hsdR); AFA47678 (Acetobacterium woodii-hsdR1); ADO36134 (Eubacterium limosum-hsdR), and the like (all of which are shown as NCBI-ProteinID).

Note here that the type I restriction enzyme is also called a type I restriction enzyme R subunit, or a type I restriction enzyme endonuclease subunit.

Examples of type I recognition enzyme (for example, EC: 3.1.21.3); ADK13414 (Clostridium ljungdahlii-hsdS); AKN31535 (Clostridium carboxidivorans-hsdS); AFA47246 (Acetobacterium woodii-hsdSI); ADO36135 (Eubacterium limosum-hsdS), and the like (all of which are shown as NCBI-ProteinID).

Note here that the type I recognition enzyme is also called a type I restriction enzyme S subunit (type I restriction enzyme, specificity subunit).

Examples of type I modification enzyme (for example, EC: 2.1.1.72): ADK13413 (Clostridium ljungdahlii-hsdM); AKN33779 (Clostridium carboxidivorans-hsdM); AFA47243 (Acetobacterium woodii-hsdM1): ADO36136 (Eubacterium limosum-hsdM), and the like (all of which are shown as NCBI-ProteinID).

Note here that the type I modification enzyme is also called a type I modification enzyme M subunit (type I restriction enzyme M protein).

In the present invention, enzymes whose enzyme activity is deleted or suppressed may be any one or two or more of the restriction enzyme, modification enzyme, and recognition enzyme. Among them, preferably, the activity of at least the restriction enzyme is deleted or suppressed. Furthermore, when the obligatory anaerobic acetogen includes a plurality of type I restriction modification system enzymes, the activity of at least one type I restriction modification system enzyme is only required to be deleted or suppressed. In addition, when a plurality of restriction enzymes, modification enzymes, or recognition enzymes are included, the activity of at least one of these is only required to be deleted or suppressed. For example, if a plurality of restriction enzymes are included, the activity of one of the restriction enzymes may be deleted or suppressed, or the activity of two or more, for example, all of the restriction enzymes may be deleted or suppressed.

As a specific example, the amino acid sequence of the type I restriction enzyme ADK13414 (hsdS) derived from Clostridium ljungdahlii is shown in SEQ ID NO: 1.

Preferably, the obligately anaerobic acetogen of the present invention can proliferate using carbon monoxide or carbon dioxide as a sole carbon source.

Furthermore, preferably, the obligately anaerobic acetogen of the present invention includes a carbon monoxide dehydrogenase. In detail, preferred is a microorganism that grows by the function of generating carbon dioxide and proton from carbon monoxide and water, mainly by carbon monoxide metabolism, that is, the action of the carbon monoxide dehydrogenase.

Furthermore, preferably, the obligately anaerobic acetogen of the present invention has a function of synthesizing acetyl-CoA from methyl tetrahydrofolate, carbon monoxide, and CoA. A pathway of synthesizing acetyl-CoA from methyl tetrahydrofolate, carbon monoxide, and CoA is included in, for example, a reduced acetyl-CoA pathway (Wood-Ljungdahl pathway) and Methanol pathway.

The obligately anaerobic acetogen of the present invention is preferably a microorganism having an acetyl-CoA pathway and further having carbon monoxide resistance and syngas assimilation property.

The obligately anaerobic acetogen of the present invention is preferably a bacterium, for example, a Clostridium bacterium or a Moorella bacterium, and particularly preferably Clostridium ljungdahlii.

Specific examples of microorganisms that can be employed as the obligately anaerobic acetogen of the present invention include Clostridium bacteria, Moorella bacteria, and Acetobacterium bacteria such as Clostridium ljungdahlii, Clostridium carboxidivorans, Moorella thermoacetica (the same as Clostridium thermoaceticum) (Pierce E G. Et al., Environ. Microbiol., 2008, vol. 10, p. 2550-2573), and Acetobacterium woodii (Dilling S. et al., Appl. Environ. Microbiol., 2007, vol. 73 (11), p. 3630-3636). These four anaerobic microorganisms are known as representative examples of the syngas assimilating microorganism. In particular, in the Clostridium bacterium, a host-vector system or a culture method has been established, and thus Clostridium bacterium is suitable as the obligately anaerobic acetogen of the present invention.

The other examples include Carboxydocella sporoducens sp. Nov. (Slepova T V. et al., Inter. J. Sys. Evol. Microbiol., 2006, vol. 56, p. 797-800), Rhodopseudomonas gelatinosa (Uffen R L, J. Bacteriol., 1983, vol. 155(3), p. 956-965), Eubacterium limosum (Roh H. et al., J. Bacteriol., 2011, vol. 193 (1), p. 307-308), Butyribacterium methylotrophicum (Lynd, L H. Et al., J. Bacteriol., 1983, vol. 153 (3), p. 1415-1423).

Note here that all of proliferation and CODH activity of the bacteria mentioned above are oxygen sensitive. However, oxygen insensitive CODH is also known. For example, oxygen insensitive CODH exists in other bacterial species represented by Bradyrhizobium japonicum (Lorite M J. Et al., Appl. Environ. Microbiol., 2000, vol. 66(5), p. 1871-1876) (King G M et al., Appl. Environ. Microbiol. 2003, 69 (12), 7257-7265). Also in Ralsotonia bacteria which are aerobic hydrogen oxidizing bacteria, oxygen insensitive CODH exists (NCBI Gene ID: 4249199, 8019399).

As described above, bacteria having CODH widely exist. The obligately anaerobic acetogen of the present invention can be appropriately selected from such bacteria. For example, using a selective medium containing CO, CO/H₂ (gas mainly containing CO and H₂), or CO/CO₂/H₂ (gas mainly containing CO, CO₂ and H₂) as a sole carbon source and energy source, a bacterium having CODH that is usable as the obligately anaerobic acetogen of the present invention can be isolated in anaerobic conditions.

The obligately anaerobic acetogen of the present invention can be cultured by a well-known method as a culture method for obligatory anaerobic microorganisms. For example, the culture can be carried out under the anaerobic conditions by using carbon sources such as saccharides, acetic acid, ethanol, methanol, and glycerine.

The present invention includes a recombinant microorganism including the above-described obligately anaerobic acetogen into which a foreign gene encoding a target protein is introduced, and being capable of expressing the target protein. Preferably, the foreign gene is incorporated into a genome.

The method of introducing a gene (DNA) into the obligately anaerobic acetogen of the present invention may be selected appropriately depending on the kind of the obligately anaerobic acetogen, and the like. For example, a vector that can be introduced into the microorganism and can allow expression of the gene incorporated therein may be used.

For example, when the microorganism is a prokaryote such as a bacterium, as the vector, a vector that can self-duplicate or can be incorporated in chromosome in a host cell, and contains a promoter at the position allowing transcription of the inserted foreign gene can be used. For example, it is preferred to construct a series of structures including a promoter, a ribosome binding sequence, the above foreign gene, and a transcription termination sequence in the microorganism by using the vector.

In the case where the obligately anaerobic acetogen is a Clostridium bacterium (including related species such as Moorella bacteria), a shuttle vector pIMP1 between Clostridium bacterium and Escherichia coli (Mermelstein L D et al., Bio/technology 1992, 10, 190-195) may be used. The shuttle vector is a fusion vector of pUC9 (ATCC 37252) and pIM13 isolated from Bacillus subtilis (Projan S J et al., J. Bacteriol. 1987, 169 (11), 5131-5139) and is retained stably in the Clostridium bacterium.

For introducing a plurality of kinds of target genes (DNA) into the obligately anaerobic acetogen of the present invention by using a vector, the genes may be incorporated in one vector, or incorporated in individual vectors. When a plurality of kinds of genes are incorporated into one vector, these genes may be expressed under a common promoter for these genes, or expressed under individual promoters. In order to express a plurality of genes in a form of a fusion protein, a fused gene in which a plurality of genes are linked to each other may be introduced.

The recombinant microorganism of the present invention can be cultured by a method well known as a culture method of an obligatory anaerobic microorganism. For example, it can be cultured using carbon sources such as saccharides, acetic acid, ethanol, methanol, and glycerin under anaerobic conditions.

The culture can be carried out using syngas. For example, when a gas component is used as a carbon source and an energy source, it is cultured, for example, in a nutrient condition including inorganic salts required for growth, and syngas. Preferably, the culture is carried out under a pressurized condition at about 0.2 to 0.3 MPa (absolute pressure). Thus, the assimilation property of the gas component is enhanced. Furthermore, for improving initial proliferation and attained cell density, small amounts of organic substances such as vitamins, yeast extract, corn steep liquor, and Bacto Tryptone, may be added.

When the recombinant microorganism of the present invention is cultured, a target protein encoded by the introduced foreign gene can be expressed. If necessary, it is possible to isolate the target protein from a cultured product.

### EXAMPLES

In the following section, the present invention will be described more specifically by way of Examples. However, the present invention is not limited to these Examples.

### (1) Construction of CRISPR-Cas9 and sgRNA Expression Vector for Deletion of Type I Restriction Enzyme

An artificially synthesized gene (6701 bp) of SEQ ID NO: 2 as a CRISPR-Cas9 and sgRNA expression gene cluster for deletion of type I restriction enzyme was constructed. The gene cluster includes Csn1 derived from Streptococcus pyogenes (endonuclease, GenBank: AAZ51387.1), each gene of a sgRNA expression cassette, and an upstream sequence (LHA, 1008 bp), a downstream sequence (RHA, 999 bp), a thI promoter (Pth1), an araE promoter (ParaE), and a fdx terminator (fdx terminator) of hsdR (type I restriction enzyme, GenBank: ADK13415.1) of Clostridium ljungdahlii, and further includes recognition sequences of NotI and XhoI at both terminals.

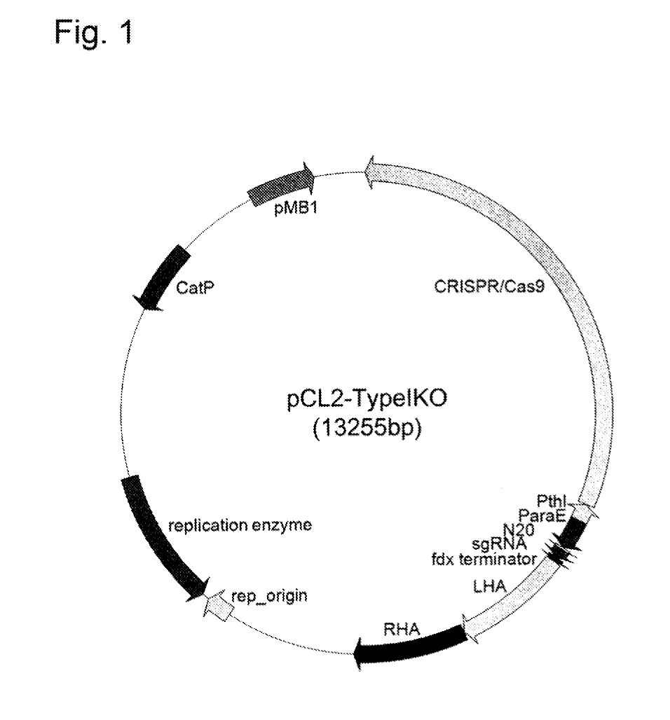

The artificially synthesized DNA (43 bp) of SEQ ID NO: 3 was cloned to the BamHI/SalI site of Clostridium/E. coli shuttle vector pCL2 to obtain pCL2-MS. Furthermore, a gene fragment of an artificially synthesized gene of SEQ ID NO: 2 that had been treated with a NotI/XhoI restriction enzyme was cloned to a NotI/XhoI site of pCL2-MS to obtain pCL2-TypeIKO (FIG. 1). In the cloning, E. coli JM109 was used. Similar to pCL2, pCL2-TypeIKO functions as a shuttle vector. The entire nucleotide sequence of pCL2-TypeIKO is shown in SEQ ID NO: 4.

### (2) Preparation of Modified Clostridium ljungdahlii in Which Type I Restriction Enzyme Activity is Deleted

pCL2-TypeIKO prepared in the above (1) was introduced into Clostridium ljungdahlii (DSM13528/ATCC55383) by electroporation to obtain a recombinant (modified Clostridium ljungdahlii). As a control, similarly, pCL2-MS was introduced into Clostridium ljungdahlii (DSM13528/ATCC55383) to obtain a recombinant. The electroporation was carried out by a method recommended in Appl. Environ. Microbiol. 2013, 79(4): 1102-9. The genomic DNA of the obtained recombinant was extracted and subjected to sequencing to confirm that the type I restriction enzyme gene had been deleted from the genome.

### (3) Confirmation of Improvement in Transformation Efficiency by Modified Clostridium ljungdahlii

Clostridium/E. coli shuttle vector pCL2 was introduced into the type I restriction enzyme deleted recombinant (modified Clostridium ljungdahlii) obtained in the above (2) and wild-type Clostridium ljungdahlii (DSM13528/ATCC55383) by electroporation, and comparison of the transformation efficiency was carried out. As a result, when the wild-type Clostridium ljungdahlii (DSM13528/ATCC55383) was used as a host, the transformation efficiency was 1.0×10² cfu/μg, while the value was 1.0×10⁵ cfu/μg when the type I restriction enzyme deleted recombinant (modified Clostridium ljungdahlii) was used. The transformation efficiency was significantly improved.

From the above, it was shown that by using the modified Clostridium ljungdahlii in which the type I restriction enzyme activity had been deleted, the transformation efficiency was improved and transformation into Clostridium ljungdahlii was able to be carried out easily.

### (4) Confirmation of Improvement in Gene Deletion Efficiency by Modified Clostridium ljungdahlii

With reference to Appl. Environ. Microbiol. 2013, 79(4): 1102-9, pUC-Δpta-ermC (SEQ ID NO: 5) including the upstream sequence and downstream sequence of a pta gene (CLJU_c12770) of C. ljungdahlii, and clarithromycin resistance gene ermC (GenBank: AB982225.1) was prepared. The pUC-Δpta-ermC was introduced into the type I restriction enzyme deleted recombinant (modified Clostridium ljungdahlii) obtained in the above (2) and the wild-type Clostridium ljungdahlii (DSM13528/ATCC55383) by electroporation, and comparison of the gene deletion efficiency was carried out. As a result, the gene deleted recombinant obtained by using the wild-type Clostridium ljungdahlii (DSM13528/ATCC55383) as a host was only one clone, while 13 clones were obtained in the case where the type I restriction enzyme deleted recombinant (modified Clostridium ljungdahlii) was used, in which the gene deletion efficiency was significantly improved.

From the above, it was shown that by using the modified Clostridium ljungdahlii in which the type I restriction enzyme activity had been deleted, the gene deletion efficiency was improved, and the genome gene of Clostridium ljungdahlii was able to be modified easily.

### (5) Confirmation of Proliferation Efficiency of Modified Clostridium ljungdahlii

The type I restriction enzyme deleted recombinant (modified Clostridium ljungdahlii) obtained in the above (2) and the wild-type Clostridium ljungdahlii (DSM13528/ATCC55383) were cultured at 37° C. under anaerobic conditions, respectively. Specifically, inoculation was carried out in 25 mL of ATCC 1754 medium (pH=5.5, fructose was not contained), and a 100 mL-volume hermetically-sealable headspace vial vessel was charged with a mixed gas of CO/CO₂/H₂=33/33/34% (volume ratio), filled with the mixed gas at a gas pressure of 0.25 MPa (absolute pressure), hermetically sealed with an aluminum cap, and then shaking culture was conducted. As a result, in the type I restriction enzyme deleted recombinant (modified Clostridium ljungdahlii), proliferation that is the same level as in the wild-type Clostridium ljungdahlii (DSM13528/ATCC5S383) was confirmed.

From the above it was shown that the type I restriction enzyme deleted mutation did not have an influence on the proliferation.

1. A modified obligately anaerobic acetogen in which activity of at least one enzyme selected from the group consisting of a restriction enzyme, a modification enzyme, and a recognition enzyme constituting a type I restriction modification system enzyme is deleted or suppressed.

2. The obligately anaerobic acetogen according to claim 1, wherein the activity of at least the restriction enzyme is deleted or suppressed.

3. The obligately anaerobic acetogen according to claim 1, wherein a part or whole of a gene encoding at least one enzyme selected from the group consisting of the restriction enzyme, the modification enzyme, and the recognition enzyme is deleted.

4. The obligately anaerobic acetogen according to claim 3, wherein a part or whole of a gene encoding at least the restriction enzyme is deleted.

5. The obligately anaerobic acetogen according to claim 1, wherein the activity of the restriction enzyme, the modification enzyme, or the recognition enzyme is deleted.

6. The obligately anaerobic acetogen according to claim 1, being capable of proliferating using carbon monoxide or carbon dioxide as a sole carbon source.

7. The obligately anaerobic acetogen according to claim 1, comprising a carbon monoxide dehydrogenase.

8. The obligatory anaerobic acetogen according to claim 1, having a function of synthesizing acetyl-CoA from methyl tetrahydrofolate, carbon monoxide, and CoA.

9. The obligately anaerobic acetogen according to claim 1, being a bacterium.

10. The obligately anaerobic acetogen according to claim 9, being a Clostridium bacterium or a Moorella bacterium.

11. The obligately anaerobic acetogen according to claim 10, being Clostridium ljungdahlii.

12. The obligately anaerobic acetogen according to claim 1, wherein the restriction enzyme is a protein including an amino acid sequence of SEQ ID NO: 1.

13. The obligately anaerobic acetogen according to claim 1, being Clostridium ljungdahlii,
wherein the activity of at least the restriction enzyme is deleted or suppressed, and
a part or whole of a gene encoding at least the restriction enzyme is deleted.

14. A recombinant microorganism comprising the obligately anaerobic acetogen according to claim 1 into which a foreign gene encoding a target protein is introduced, and being capable of expressing the target protein.

15. The recombinant microorganism according to claim 14, wherein the foreign gene is incorporated into a genome.
